(19) United States
(12) Patent Application Publication
(10) Pub. No.: US 2026/0243703 A1
(43) Pub. Date: Aug. 20, 2026

(54) SAFETY HOOKS FOR CEILING MOUNTED MEDICAL IMAGING SYSTEM

(71) Applicant: GE Precision Healthcare LLC, Waukesha, WI (US)

(72) Inventors: Weidong Lv, Beijing (CN); Julien Marcotte, Saint Vrain (FR); Romain Chatelin, Plaisir (FR); Guoli Pan, Beijing (CN); Xiaoyi Guo, Beijing (CN); Jianwei Liu, Beijing (CN)

(21) Appl. No.: 19/057,340

(22) Filed: Feb. 19, 2025

(51) Int. Cl. G01N 23/04

(52) U.S. Cl. CPC G01N 23/04; G01N 2223/1016; G01N 2223/308

(57) ABSTRACT

A ceiling mount includes a nut holding a bottom of an upper protrusion of a ceiling rail, a ceiling safety hook, a screw, and a chassis. The ceiling safety hook includes an upper mount fastened to a strut in the ceiling and a lower mount extending downward from the upper mount, surrounding the nut, and extending below the upper protrusion. The screw extends through the upper mount and the nut to secure the ceiling rail to the strut. The lower mount receives the bottom side of the upper protrusion in a failure condition. The chassis is coupled to the medical imaging system and includes a linear guide assembly having rollers slidably coupled to a lower protrusion of the ceiling rail and a chassis safety hook extending above a top side of the lower protrusion. The chassis safety hook receives the top side of the lower protrusion in a failure condition.

### FIELD

Certain embodiments relate to ceiling mounts for medical imaging systems. More specifically, certain embodiments relate to safety hooks for a ceiling mounted medical imaging system that prevents the ceiling mounted medical imaging system from falling if a screw or other attachment mechanism of the mounting assembly fails.

### BACKGROUND

Medical diagnostic imaging systems generate images of a patient through exposure to an energy source, such as X-rays passing through a patient, for example. The generated images may be used to provide a medical diagnosis of a patient, among other things. In some cases, the medical diagnostic imaging system may be mounted to a ceiling in an examination room. The medical diagnostic imaging system may be movable and rotatable to acquire medical image data from different positions and/or angles. One example of a ceiling mounted medical diagnostic imaging system is C-arm X-ray diagnostic equipment. The term C-arm generally refers to an X-ray imaging device having a rigid and/or articulating structural member having an X-ray source and an image detector assembly that are each located at an opposing end of the structural member so that the X-ray source and the image detector face each other. The structural member is typically “C” shaped and so is referred to as a C-arm. In this manner, X-rays emitted from the X-ray source can impinge on the image detector and provide an X-ray image of the object or objects that are placed between the X-ray source and the image detector. The weight of a C-arm X-ray diagnostic medical imaging system may be 800 kilograms or more. If a screw or other attachments mechanism of the mounting assembly were to loosen or break and the C-arm X-ray diagnostic medical imaging system were to fall on a patient, the result could be catastrophic.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

Safety hooks for a ceiling mounted medical imaging system that prevent the ceiling mounted medical imaging system from falling if a screw or other attachment mechanism of the mounting assembly fails is provided, substantially as shown in and/or described in connection with at least one of the figures, as set forth more completely in the claims.

These and other advantages, aspects, and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram illustrating components of an exemplary X-ray imaging system, in accordance with various embodiments.

FIG. 2 is a schematic diagram of a side view of an exemplary X-ray imaging system mounted to a ceiling, in accordance with various embodiments.

FIG. 3 is a schematic diagram of a lateral view of an X-ray imaging system mounted to a ceiling by a lower ceiling mount and an upper ceiling mount, in accordance with various embodiments.

FIG. 4 is a schematic diagram of a lateral view of an X-ray imaging system mounted to a ceiling by a lower ceiling mount and an upper ceiling mount, in accordance with various embodiments.

FIG. 5 is a schematic diagram of a perspective view of a portion of an upper ceiling mount having a chassis mounted to rails of a rail system and the rails mounted to struts of a ceiling grid by a ceiling rail safety hook and a rail fixed nut, in accordance with various embodiments.

FIG. 6 is a schematic diagram of a lateral view of a ceiling rail mounted to a strut of a ceiling grid by the ceiling rail safety hook and rail fixed nut identified in FIG. 4, in accordance with various embodiments.

FIG. 7 is a schematic diagram of a top perspective view of a ceiling rail mounted to a strut of a ceiling grid by a ceiling rail safety hook and rail fixed nut, in accordance with various embodiments.

FIG. 8 is a schematic diagram of a bottom perspective view of a ceiling rail mounted to a strut of a ceiling grid by a ceiling rail safety hook and rail fixed nut, in accordance with various embodiments.

FIG. 9 is a schematic diagram of a lateral view of ceiling rail safety hooks with rail fixed nuts and mounting screws provided on opposite sides of a ceiling rail to mount the ceiling rail to a strut of a ceiling grid in a normal condition, in accordance with various embodiments.

FIG. 10 is a schematic diagram of a lateral view of ceiling rail safety hooks provided on opposite sides of a ceiling rail to prevent the ceiling rail from falling from a strut of a ceiling grid after failure of the rail fixed nuts and/or mounting screws shown in FIG. 9, in accordance with various embodiments.

FIG. 11 is a schematic diagram of a perspective view of a portion of an upper ceiling mount having a chassis beams slidably mounted to rails of a rail system by linear guide assemblies having chassis safety hooks, in accordance with various embodiments.

FIG. 12 is a schematic diagram of a perspective view of a linear guide assembly slidably attached to a lower protrusion of a ceiling rail by rollers, the linear guide assembly having chassis safety hooks, in accordance with various embodiments.

FIG. 13 is a schematic diagram of a lateral view of a linear guide assembly slidably attached to a lower protrusion of a ceiling rail by rollers in a normal condition, the linear guide assembly having chassis safety hooks, in accordance with various embodiments.

FIG. 14 is a schematic diagram of a lateral view of a linear guide assembly having chassis safety hooks to prevent a chassis attached to the linear guide assembly from falling from the ceiling rail after failure of the upper rollers shown in FIG. 13, in accordance with various embodiments.

### DETAILED DESCRIPTION

Certain embodiments may be found in ceiling rail safety hooks and chassis safety hooks for a ceiling mounted medical imaging system that prevents the ceiling mounted medical imaging system from falling if a mounting screw, rail fixed nut, roller, or other attachment mechanism of the mounting assembly fails. For example, aspects of the present disclosure have the technical effect of ensuring the safety of patients and operators by substantially reducing and/or eliminating the risk of a ceiling mounted medical imaging system falling if a mounting screw, rail fixed nut, roller, or other attachment mechanism of the mounting assembly fails. In various embodiments, the ceiling rail safety hooks and chassis safety hooks take no load during normal operation. Instead, the ceiling rail safety hooks and chassis safety hooks carry the load of the medical imaging system only if a failure occurs.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical, and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

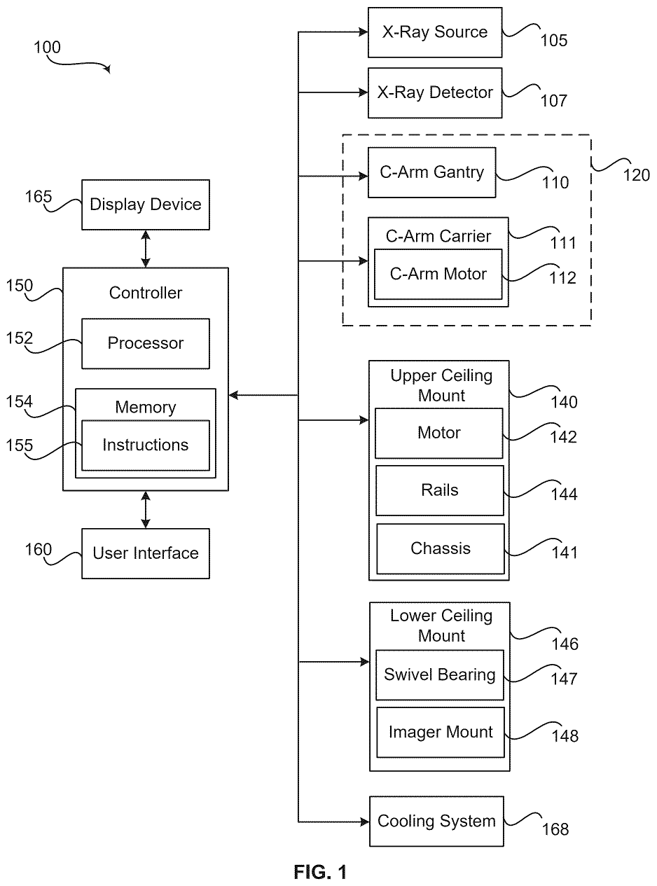

FIG. 1 is a block diagram illustrating components of an exemplary X-ray imaging system 100, in accordance with various embodiments. Referring to FIG. 1, there is shown an X-ray imaging system 100 that comprises a controller 150, a user interface 160, a display device 165, an X-ray source 105, and X-ray detector 107, a gantry structure 120, an upper ceiling mount 140, a lower ceiling mount 146, and a cooling system 168, among other things. In certain embodiments, the X-ray imaging system 100 is configured to perform vascular imaging, such as digital subtraction angiography (DSA). The X-ray imaging system 100 is configured to be mounted to a ceiling. In various embodiments, the X-ray imaging system 100 is mounted to the ceiling at a fixed location. In certain embodiments, the X-ray imaging system 100 is mounted to the ceiling but may move along the ceiling (e.g., via chassis that moves along a rail system). The X-ray imaging system 100 includes an X-ray source 105 (or X-ray radiation source) and an X-ray detector 107 mounted on a C-arm gantry 110 (e.g., C-arm). In an exemplary embodiment, the C-arm gantry 110 may be made of carbon fiber.

The C-arm gantry 110 is part of a gantry structure 120. The gantry structure 120 includes a C-arm motor 112 for adjusting the position of the C-arm gantry 110. More specifically, the C-arm gantry 110 is mechanically coupled to a C-arm carrier 111 (e.g., C-arm rotation device) which includes the C-arm motor 112, and the C-arm motor 112 may be driven to adjust the position of the C-arm gantry 110 with respect to the C-arm carrier 111. For example, the C-arm carrier 111 in conjunction with the C-arm motor 112 is configured to rotate the C-arm gantry 110 in an orbital direction relative to the C-arm carrier 111. In certain embodiments, the C-arm carrier 111 (via a motorized system) is configured to rotate a pivot (e.g., pivot point) where the C-arm carrier 111 is coupled to an end of an arm (e.g., L-arm) coupled or mounted to the ceiling. The C-arm carrier 111 rotates about a rotational axis (e.g., horizontal axis) of the pivot. In representative embodiments having an L-arm, the L-arm may rotate about a location where the other end of the L-arm (i.e., the end of the L-arm not connected to the pivot) is coupled to or mounted to the ceiling (via a lower ceiling mount 146). In various embodiments, the C-arm carrier 111 is coupled to or mounted to the ceiling (via the lower ceiling mount 146).

The upper ceiling mount 140 is coupled to a ceiling. In certain embodiments, the C-arm carrier 111 is coupled to the upper ceiling mount 140 via an L-arm (e.g., via the end of the L-arm not connected to the pivot) coupled to the lower ceiling mount 146. The upper ceiling mount 140 is configured to move (e.g., translocate) the X-ray imaging system 100 from one location to another location (e.g., in a linear direction) on the ceiling. The upper ceiling mount 140 includes a chassis 141. The upper ceiling mount 140 also includes a motor 142 and a rail system 144 (e.g., having rails). The rail system 144 is directly coupled to the ceiling. For example, ceiling rails of the rail system 144 may be mounted to struts of a ceiling grid by rail fixed nuts and mounting screws. In a representative embodiment, ceiling rail safety hooks are provided to prevent the ceiling rails of the rail system 144 from falling from the struts of the ceiling grid if the rail fixed nuts and/or mounting screws fail, as discussed in more detail below. The motor 142 is configured to drive movement of the chassis 141 and, thus, the X-ray imaging system 100 along the rail system 144 (e.g., to adjust a position of the X-ray imaging system 100). Accordingly, the chassis 141 may be slidably mounted by linear guide assemblies to the ceiling rails of the rail system 144. In various embodiments, the linear guide assemblies comprise rollers for slidably mounting the chassis 141 to the ceiling rails. In an exemplary embodiment, chassis safety hooks are provided on the linear guide assemblies to prevent the chassis 141 from falling from the ceiling rails of the rail system 144 if the rollers fail, as discussed in more detail below.

The lower ceiling mount 146 is configured to rotatably couple the gantry structure 120 to the upper ceiling mount 140. In various embodiment, the lower ceiling mount 146 may be operable to dampen vibrations that occur when the C-arm gantry 110 (e.g., made of carbon fiber) moves and/or deaccelerates (e.g., during the deacceleration phase) to a stop during movement of the gantry structure 120 along multiple axes or directions (e.g., combined directions). For example, the movement of the gantry structure 120 may include the rotational movement of the C-arm gantry 110 in the orbital direction, rotational movement about the axis of the pivot where the C-arm carrier 111 is coupled to an end of an arm (e.g., L-arm), and/or rotation about where an axis of where the lower ceiling mount 146 (e.g., axis of the swivel bearing 147) is coupled the ceiling. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction extending between a floor and the ceiling and a horizontal direction that is both perpendicular to the vertical direction and parallel with the floor.

The lower ceiling mount 146 includes a swivel bearing 147 and an imager mount 148. The imager mount 148 includes a body having a first end and a second end opposite the first end, a first mount connection disposed on the first end, and a second mount connection coupled to the second end. The first mount connection and the second mount connection both directly contact both the gantry structure 120 and the body. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

The swivel bearing 147 pivotally couples the gantry structure 120 to the upper ceiling mount 140 (e.g., the chassis) when the X-ray imaging system 100 is mounted to the ceiling. The swivel bearing 147 is disposed on a side of the body facing the ceiling. In certain embodiments, the body is made of a material that is stiffer than both the first mount connection and the second mount connection. The swivel bearing 147 is configured to enable the gantry structure 120 to rotate 360 degrees about a rotational axis of the swivel bearing 147.

The X-ray imaging system 100 further includes a controller 150 comprising suitable logic, circuitry, interfaces, and/or code that may be operable to control operation of the X-ray imaging system 100. The controller 150 comprises a processor 152 and a non-transitory memory 154. A method for controlling the X-ray imaging system 100 may be stored as executable instructions 155 in the non-transitory memory 154 and executed by the processor 152.

The memory 154 may be one or more computer-readable memories integrated with the X-ray imaging system 100 and/or communicatively coupled (e.g., over a network) to the X-ray imaging system 100, such as a Picture Archiving and Communication System (PACS), a server, a hard disk, floppy disk, CD, CD-ROM, DVD, compact storage, flash memory, random access memory, read-only memory, electrically erasable and programmable read-only memory, optical storage medium, magnetic storage medium, solid-state storage medium, and/or any suitable memory. The memory 154 may include databases, libraries, sets of information, or other storage accessed by and/or incorporated with the processor 152, for example. The memory 154 may be able to store data temporarily or permanently, for example. The memory 154 may store processor-executable software code or instructions (e.g., firmware or software) 155, which are tangibly stored on a non-transitory computer readable medium. Additionally or alternatively, the memory 154 may store medical image data, data generated by the processor 152, and/or any suitable data.

The processor 152 may comprise suitable logic, circuitry, interfaces and/or code that may be operable to process medical image data for generating medical images (e.g., X-ray images) for presentation on a display device 165. The processor 152 is operable to perform one or more processing operations on the acquired medical image data. In an exemplary embodiment, the processor 152 may be operable to perform display processing and/or control processing, among other things. Acquired medical image data may be processed in real-time during an imaging examination as the medical image data is received. Additionally or alternatively, the medical image data may be stored temporarily during an imaging examination and processed in less than real-time in a live or off-line operation. In various embodiments, the processed image data can be presented at the display device 165 and/or may be stored at the memory 154. The memory 154 may be a local archive, a Picture Archiving and Communication System (PACS), or any suitable device for storing images and related information. The processor 152 may be one or more central processing units, graphic processing units, microprocessors, microcontrollers, and/or the like. The processor 152 may be an integrated component, or may be distributed across various locations, for example. In an exemplary embodiment, the processor 132 may be capable of receiving input information from a user interface 160 and/or memory 154, generating an output displayable by a display device 165, and manipulating the output in response to input information from a user interface 160, among other things. The processor 152 may be capable of executing any of the method(s) and/or set(s) of instructions 155 discussed herein in accordance with the various embodiments, for example.

The user interface 160 may be utilized to receive input from a user or operator of the X-ray imaging system 100. The user interface 160 may be communicatively coupled to the controller 150 for providing commands input by a user via the user interface 160 to the controller 150. For example, the user interface 160 may receive inputs of patient data, image acquisition parameters, settings, configuration parameters, select protocols and/or templates, and the like. In an exemplary embodiment, the user interface 160 may be operable to configure, manage and/or control operation of one or more components and/or modules in the X-ray imaging system 100. In this regard, the user interface 160 may be operable to configure, manage and/or control operation of the controller 150, user interface 160, display device 165, X-ray source 105, X-ray detector 107, C-arm gantry 110, C-arm carrier 111, upper ceiling mount 140, lower ceiling mount 146 and/or the cooling system 168. The user interface 160 may include hard button(s), soft button(s), rotary encoder(s), a touchscreen, motion tracking, voice recognition, a mousing device, keyboard, trackball, joystick(s), a touchpad, camera, and/or any other device capable of receiving a user directive. In certain embodiments, one or more of the user interfaces 160 may be integrated into other components, such as the display device 165, for example. As an example, user interface 160 may include a touchscreen display. In some examples the user interface 160 may be remotely located relative to the X-ray imaging system 100. For example, the user interface 160 may be communicatively coupled to the controller 150 and/or the X-ray imaging system 100 via a wired or wireless connection.

As an example, a user of the X-ray imaging system 100 may input a desired isocenter position via the user interface 160. The controller 150 may then determine position adjustments to one or more of the C-arm gantry 110 and/or the upper ceiling mount 140 to align an isocenter of the X-ray imaging system 100 with the desired isocenter position. As another example, a user of the X-ray imaging system 100 may directly control the position of one or more components of the X-ray imaging system 100 relative to other components of the X-ray imaging system 100 via the user interface 160. For example, the user may directly input, via a joystick or knob, for example, position adjustments to one or more components of the X-ray imaging system 100. As another example, the motion of the components of the X-ray imaging system 100 may be pre-programmed such that the user does not directly control any movement, but instead initiates the start of the pre-programmed motion. The motion may include complex motions, with continuous motion of the isocenter.

The controller 150 is further communicatively coupled to a display device 165 for displaying one or more X-ray images acquired via the X-ray detector 107. Further, in some examples, one or more of the controller 150, the user interface 160, and the display device 165 may be positioned away from (e.g., remotely from) the remaining components of the X-ray imaging system 100. The display device 165 may be any device capable of communicating visual information to a user. For example, a display device 165 may include a liquid crystal display, a light emitting diode display, and/or any suitable display or displays.

The X-ray imaging system 100 may further include a cooling system 168 for cooling the X-ray source 105 and/or the X-ray detector 107. The cooling system 168 may include one or more flexible tubes and a pump, as an illustrative and non-limiting example, providing cooling fluid to the X-ray source 105 to transfer thermal energy away from the X-ray source 105. The cooling system 168 may actively cool the X-ray source 105 and the X-ray detector 107 independently, or in some examples may cool the X-ray detector 107 by any suitable type of derivation of the cooling circuit for the X-ray source 105.

Components of the X-ray imaging system 100 may be implemented in software, hardware, firmware, and/or the like. The various components of the X-ray imaging system 100 may be communicatively linked. Components of the X-ray imaging system 100 may be implemented separately and/or integrated in various forms. For example, the display device 165 and the user input device 130 may be integrated as a touchscreen display.

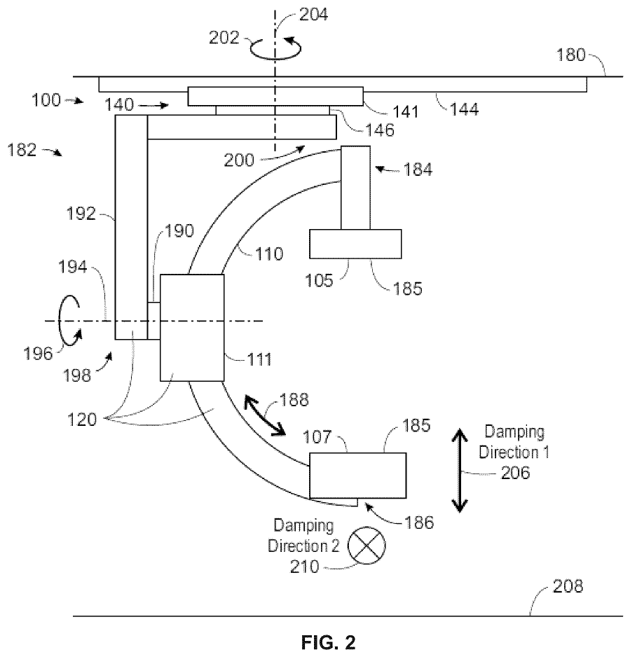

FIG. 2 is a schematic diagram of a side view of an exemplary X-ray imaging system 100 mounted to a ceiling 180, in accordance with various embodiments. The X-ray imaging system 100 of FIG. 2 shares various characteristics with the X-ray imaging system of FIG. 1 as described above. Referring to FIG. 2, the X-ray imaging system 100 is provided in an examination room 182 having a ceiling 180 and a floor 208. The X-ray imaging system 100 comprises an X-ray source 105, and X-ray detector 107, a gantry structure 120 having a C-arm gantry 110, a C-arm carrier 111 and a mounting structure 192, an upper ceiling mount 140, and a lower ceiling mount, among other things.

The gantry structure 120 comprises a C-arm gantry 110, a C-arm carrier 111, and a mounting structure 192. In certain embodiments, the C-arm gantry 110 is made of carbon fiber. The X-ray radiation source 105 is coupled to a first end 184 of the C-arm gantry 110 and the X-ray detector 107 is coupled to a second end 186 of the C-arm gantry 110 opposite the first end 184 (e.g., forming the image chain 185). The C-arm gantry 110 is coupled to the C-arm carrier 111 (e.g., C-arm rotation device), which is configured to rotate the C-arm gantry 110 in an orbital direction 188 relative to the C-arm carrier 111 about an isocenter of the X-ray radiation source 105 and the X-ray detector 107. The C-arm carrier 111 includes rollers (e.g., guiding rollers) to guide movement of the C-arm gantry 110 relative to the C-arm carrier 111.

The C-arm carrier 111 is coupled to a pivot 190 (e.g., pivot point or shaft). The pivot 190 is coupled to the mounting structure 192. The pivot 190 is driven by a motor (e.g., C-arm motor or any suitable motor) configured to rotate both the C-arm carrier 111 and the C-arm gantry 110 about a rotational axis 194 (e.g., horizontal axis) of the pivot 190 as indicated by arrow 196. In certain embodiments, the mounting structure 192 is an L-arm coupled to the upper ceiling mount 140 via the lower ceiling mount 146. The pivot 190 is coupled to a first end 198 of the L-arm 192 and the lower ceiling mount 146 is coupled to a second end 200 of the L-arm 192. The lower ceiling mount 146 is configured to couple the gantry structure 120 to the upper ceiling mount 140 (and, thus, mount the X-ray imaging system 100 to the ceiling 180). In certain embodiments, the L-arm may rotate about an end of the L-arm coupled to the upper ceiling mount 140 via the swivel bearing of the lower ceiling mount 146. In particular, the L-arm (and the gantry structure 120) rotate in direction 202 about a rotational axis 204 of the swivel bearing.

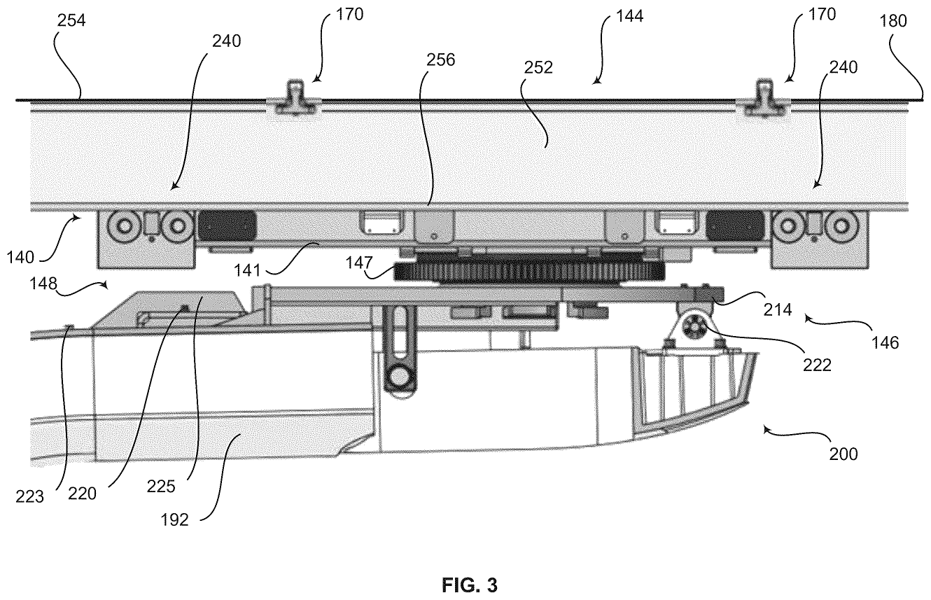

The C-arm carrier 111 is coupled to the upper ceiling mount 140 via the L-arm 192 (e.g., via the end 200 of the L-arm not connected to the pivot 190) coupled to the lower ceiling mount 146. The upper ceiling mount 140 is coupled to the ceiling 180. The upper ceiling mount 140 is configured to linearly translate the X-ray imaging system 100 from one location to another location across the ceiling 180. The upper ceiling mount 140 includes a chassis 141, a motor 142, and a rail system 144 (e.g., having rails). The rail system 144 is directly coupled to the ceiling 180. For example, ceiling rails of the rail system 144 may be mounted to struts of a ceiling grid by rail fixed nuts and mounting screws. In a representative embodiment, ceiling rail safety hooks are provided to prevent the ceiling rails of the rail system 144 from falling from the struts of the ceiling grid if the rail fixed nuts and/or mounting screws fail, as discussed in more detail below with reference to FIGS. 3-10. In various embodiments, the chassis 141 is slidably mounted by linear guide assemblies to the ceiling rails of the rail system 144. In certain embodiments, the linear guide assemblies comprise rollers for slidably mounting the chassis 141 to the ceiling rails. In an exemplary embodiment, chassis safety hooks are provided on the linear guide assemblies to prevent the chassis 141 from falling from the ceiling rails of the rail system 144 if the rollers fail, as discussed in more detail below with reference to FIGS. 3, 4 and 11-14. The motor 142 is configured to drive movement of the chassis 141 and, thus, the X-ray imaging system 100 along the rail system 144 (e.g., to adjust a position of the X-ray imaging system 100).

The lower ceiling mount 146 is coupled to the upper ceiling mount 140. In particular, a swivel bearing of the lower ceiling mount 146 is rotatably coupled to a chassis 141 of the upper ceiling mount 140. In various embodiments, the lower ceiling mount 146 is configured to dampen vibrations caused by motion of the gantry structure 120 for an entirety of the gantry structure 120. For example, the movement of the gantry structure 120 may include the rotational movement of the C-arm gantry 110 in the orbital direction 188, rotational movement in the direction 196 about the axis 194 of the pivot 190 where the C-arm carrier 111 is coupled to an end of the mounting structure 192 (e.g., L-arm), and/or rotational movement in the direction 202 about the 204 axis of where the lower ceiling mount 146 (e.g., axis 204 of the swivel bearing) is coupled to the ceiling 180. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction 206 extending between a floor 208 and the ceiling 180 and a horizontal direction 210 (into the page in FIG. 2) that is both perpendicular to the vertical direction 206 and parallel with the floor 208.

The lower ceiling mount includes a swivel bearing (e.g., which couples the gantry structure to the chassis 141 of the upper ceiling mount 140 installed on the ceiling 180) and an imager mount. The swivel bearing is disposed on a side of the body facing the ceiling 180. The imager mount includes a body having a first end and a second end opposite the first end, a first mount connection disposed on the first end, and a second mount connection coupled to the second end. The first mount connection and the second mount connection both directly contact both the mounting structure 192 of the gantry structure 120 and the body. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

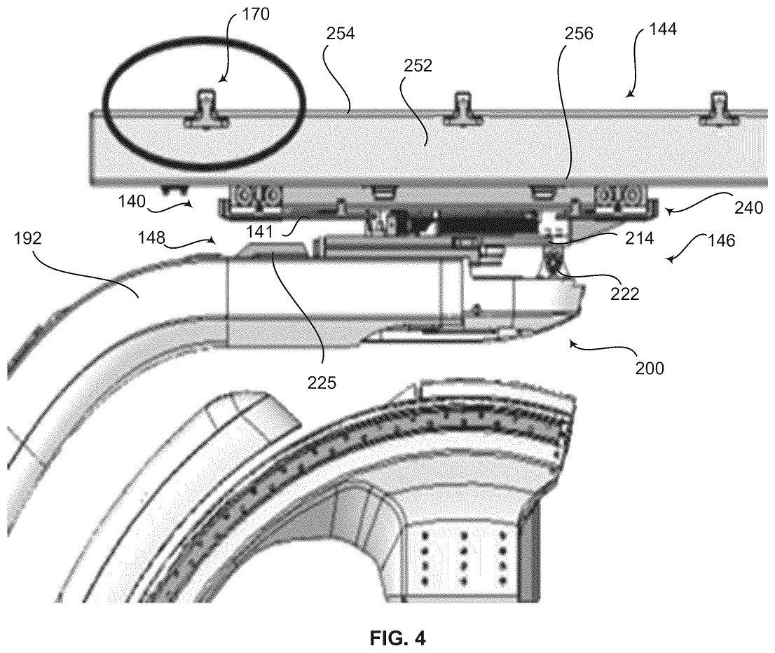

FIGS. 3 and 4 are schematic diagrams of a lateral view of an X-ray imaging system 100 mounted to a ceiling 180 by a lower ceiling mount 146 and an upper ceiling mount 140, in accordance with various embodiments. The upper ceiling mount 140 and lower ceiling mount 146 for mounting a mounting structure 192 (e.g., L-arm) of a medical imaging system (e.g., an X-ray system 100) to a ceiling 180 as shown in FIGS. 3 and 4 share various characteristics with the upper ceiling mount 140 and lower ceiling mount 146 for mounting a mounting structure 192 (e.g., L-arm) of a medical imaging system (e.g., an X-ray system 100) to a ceiling 180 described above with reference to FIGS. 1 and 2.

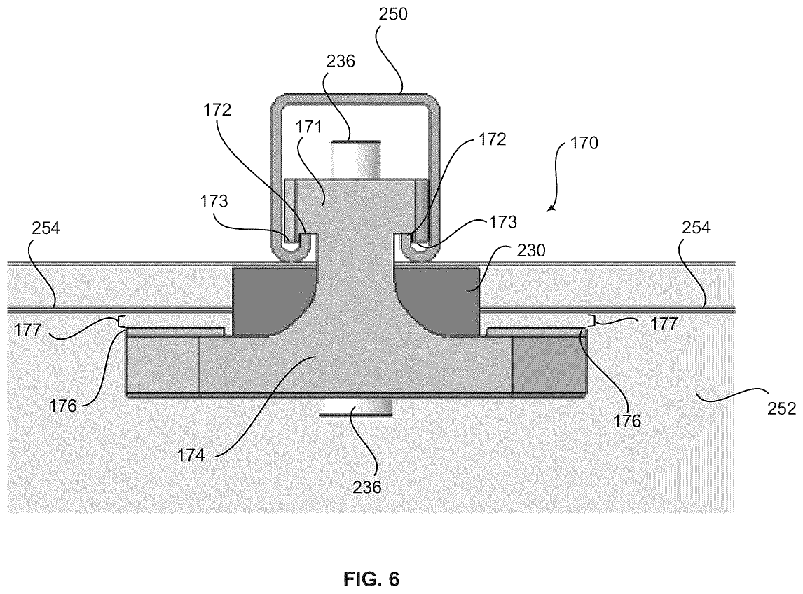

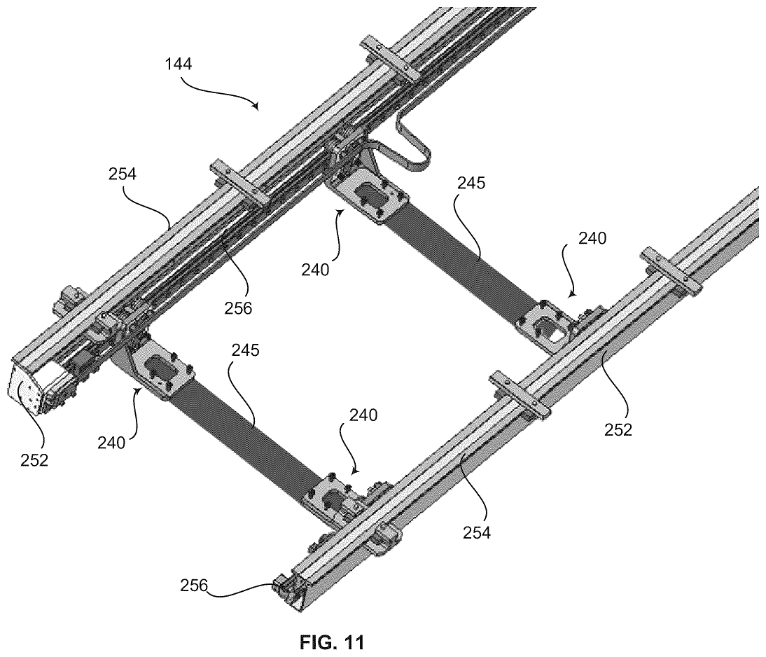

Referring to FIGS. 3 and 4, a lower ceiling mount 146 is coupled to a mounting structure 192 of a medical imaging system, such as a gantry structure of an X-ray imaging system. Only a portion of the mounting structure 192 is shown in FIGS. 3 and 4. In various embodiments, the portion of the mounting structure 192 may be an L-arm 192 of a gantry structure 120 as shown in FIGS. 1 and 2. The lower ceiling mount 146 is configured to be rotatably coupled to an upper ceiling mount 140 installed on the ceiling 180. The upper ceiling mount 140 is configured to rectilinearly move the medical imaging system (e.g., X-ray imaging system 100) from one location to another location across the ceiling 180. The upper ceiling mount 140 comprises a chassis 141, a rail system 144, and a motor. The rail system 144 comprises parallel rails 252 coupled to struts of a ceiling grid in the ceiling 180. The parallel rails 252 include upper protrusions 254 extending horizontally from both sides of each of the rails 252. The parallel rails 252 comprise lower protrusions 256 extending horizontally from at least an interior side (i.e., the side facing the corresponding parallel rail 252) of each of the rails 252. The upper protrusions 254 on both sides of each of the parallel rails 252 are secured to struts of a ceiling grid in the ceiling 180 by rail fixed nuts and mounting screws. In an exemplary embodiment, ceiling rail safety hooks 170 are provided with rail fixed nuts and mounting screws to prevent the ceiling rails 252 from falling from the ceiling 180 if the rail fixed nuts and/or mounting screws fail (i.e., a failure condition). A detailed view of the ceiling rail safety hook 170 with rail fixed nut and mounting screw as identified in FIG. 4 is illustrated in FIGS. 6-10, for example. The chassis 141 comprises linear guide assemblies 240 having rollers that slidably couple the chassis 141 to the lower protrusions 256 extending from the interior of the rails 252 of the rail system 144. In various embodiments, the linear guide assemblies 240 comprises chassis safety hooks to prevent the chassis 141 from falling from the ceiling rails 252 of the rail system 144 if the rollers fail (i.e., a failure condition), as discussed in more detail below with reference to FIGS. 11-14. The motor is operable to drive movement of the chassis 141 linearly along the ceiling rails 252 to adjust a position of the medical imaging system (e.g., X-ray imaging system 100).

The lower ceiling mount 146 includes a swivel bearing 147 and an imager mount 148. A bottom side of the swivel bearing 147 is disposed on a top side 224 of a body 214 of the imager mount 148 facing the ceiling 180. A top side of the swivel bearing 147 may be rotatably coupled to the chassis 141 of the upper ceiling mount 140. For example, the swivel bearing 147 may comprise a fixed outer ring attached to the chassis 141 and a pivotable inner ring coupled to the imager mount 148. A motor may drive rotation of the inner ring of the swivel bearing 147 to adjust an orientation of the medical imaging system (e.g., X-ray imaging system 100) by rotating the imager mount 148 to a desired position. The swivel bearing 147 is configured to enable the medical imaging system (e.g., X-ray imaging system 100) to rotate 360 degrees in the direction 202 about the rotational axis 204 of the swivel bearing 147.

The imager mount 148 includes a body 214 having a first end 216 and a second end 218 opposite the first end 216, a first mount connection 220, 223, 225 disposed on the first end 216, and a second mount connection 222 coupled to the second end 218. As shown in FIGS. 3 and 4, both the first mount connection 220, 223, 225 and the second mount connection 222 directly contact both the mounting structure 192 and the body 214. In certain embodiments, the first mount connection 220, 223, 225 includes a mounting bracket 225 attached at a first end to the body 214 by attachment mechanisms 220, such as one or more damping pads or dampers (e.g., made of an elastomeric material such as rubber) and mounting screws or any suitable attachment mechanisms. A second end of the mounting bracket 225 is attached to the mounting structure 192 by attachment mechanisms 223, such as mounting screws or any suitable attachment mechanisms. The first mount connection 220, 223, 225 is disposed on a top side 224 of the body 214 (e.g., facing the ceiling 180). In particular, the first mount connection 220, 223, 225 is disposed between and contacts the mounting structure 192 and the body 214. The second mount connection 222 includes a shaft and bushings coupled to opposite ends of the shaft. The shaft extends through a portion of a second end 200 of the mounting structure 192 and portions of the body 214 located on a bottom side (e.g., facing away from the ceiling 180) of the body 214. In various embodiments, the body 214 is made of a material that is stiffer than both the first mount connection 220, 223, 225 and the second mount connection 222. The stiffer body 214 minimizes the movement of the gantry structure 120 during damping of vibrations.

In various embodiments, the lower ceiling mount 146 is configured to dampen vibrations caused by motion of the medical imaging system (e.g., a gantry structure 120 of an X-ray imaging system 100) for an entirety of the medical imaging system along the multiple directions. The multiple directions may include orthogonal directions. For example, the orthogonal directions may include a vertical direction 206 (damping direction 1) extending between the floor and the ceiling 180 and the horizontal direction 210 (damping direction 2) that is both perpendicular to the vertical direction 206 and parallel with the floor 208 as depicted in FIG. 2. As depicted in FIG. 2, the damping directions 1 and 2 may include a circumferential element, such as about a rotational axis of the shaft and side to side movement of lateral sides of the lower ceiling mount 146, respectively.

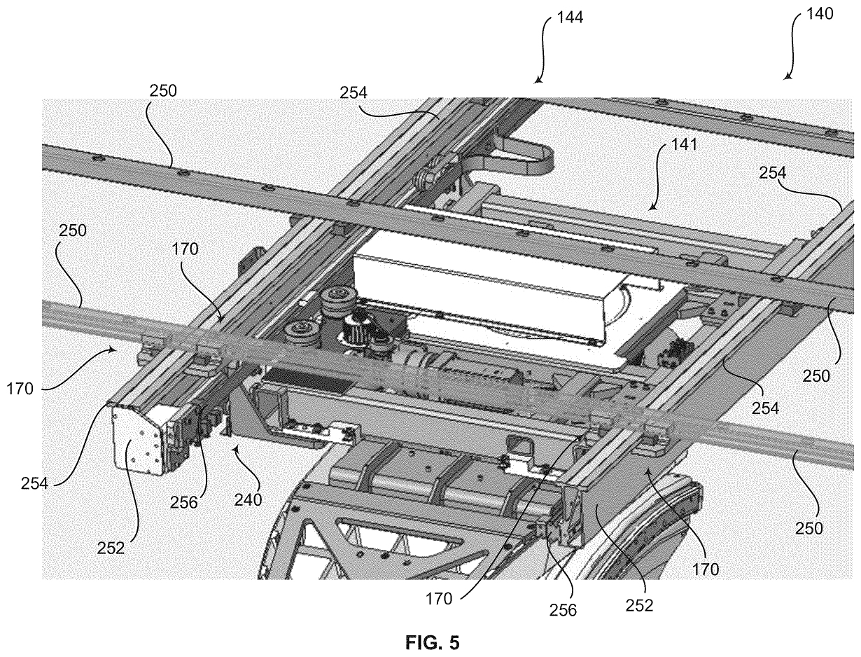

FIG. 5 is a schematic diagram of a perspective view of a portion of an upper ceiling mount 140 having a chassis 141 mounted to rails 252 of a rail system 144 and the rails 252 mounted to struts 250 of a ceiling grid by ceiling rail safety hooks 170 and a rail fixed nut, in accordance with various embodiments. Referring to FIG. 5, a ceiling grid formed by struts 250 is shown. The struts 250 may be UNISTRUT or any suitable struts having channels configured to receive fixture mountings. The struts 250 are arranged with the channel facing the floor such that fixture mountings may be inserted into the channel and supported by a lip within the channel of the strut 250. Still referring to FIG. 5, two parallel ceiling rails 252 of a rail system 144 of an upper ceiling mount 140 are shown mounted to the struts 250 by rail fixed nuts and mounting screws. In a preferred embodiment, ceiling rail safety hooks 170 are provided with the rail fixed nuts and mounting screws to prevent the ceiling rails 252 from falling from the struts 250 if the rail fixed nuts and/or mounting screws fail (i.e., a failure condition). A detailed view of the ceiling rail safety hook 170 with rail fixed nut and mounting screw is illustrated in FIGS. 6-10, for example. The rail fixed nuts are provided at upper protrusions 254 extending horizontally outward from both sides of the ceiling rails 252 at the top of the ceiling rails 252. The rail fixed nuts partially extend under the upper protrusions 254 to carry the load of the ceiling rails 252 along with the medical imaging system (e.g., X-ray imaging system 100) installed on the ceiling mounts 140, 146. The rail fixed nuts are attached to the struts 250 by the mounting screws extending through the rail fixed nuts and into upper mount portions of the ceiling mount safety hooks 170 installed over the lip in the channels of the struts 250 as described in more detail below with reference to FIGS. 6-9. The ceiling rails 252 further comprise lower protrusions 256 extending from interior sides of the two parallel ceiling rails 252. A chassis 141 of the upper ceiling mount 140 is slidably coupled to the lower protrusions 256 of the ceiling rails 252 by linear guide assemblies 240 provided at the four corners of the chassis 141. The linear guide assemblies 240 comprise rollers operable to slidably engage with the lower protrusions 256 of the ceiling rails 252. In a preferred embodiment, the linear guide assemblies 240 comprise chassis safety hooks to prevent the chassis 141 from falling from the ceiling rails 252 of the rail system 144 if the rollers fail (i.e., a failure condition), as discussed in more detail below with reference to FIGS. 11-14. The chassis 141 of the upper ceiling mount 140 is pivotally coupled to a lower ceiling mount by a swivel bearing, for example. A medical imaging system, such as an X-ray imaging system 100, may be installed on the lower ceiling mount.

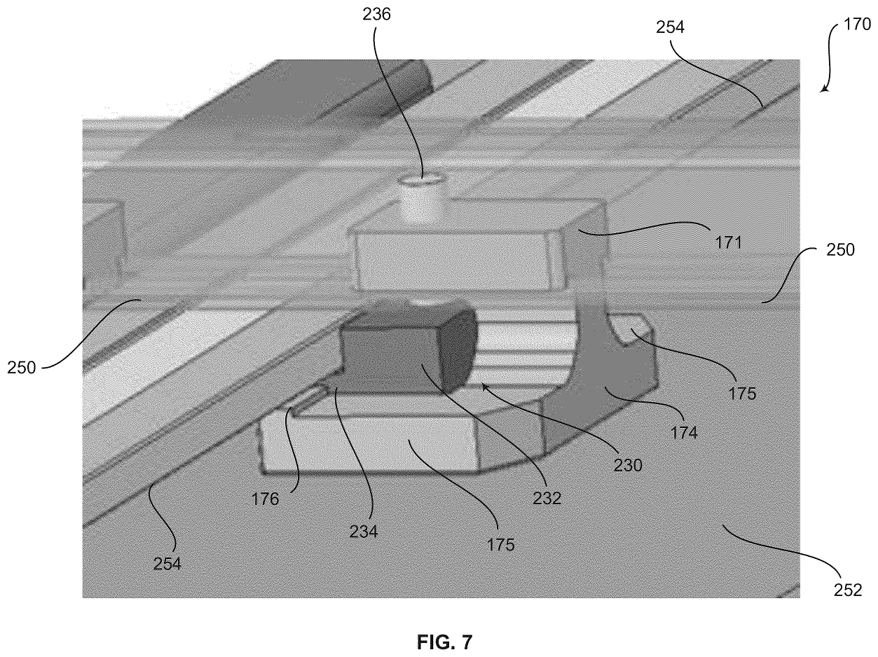

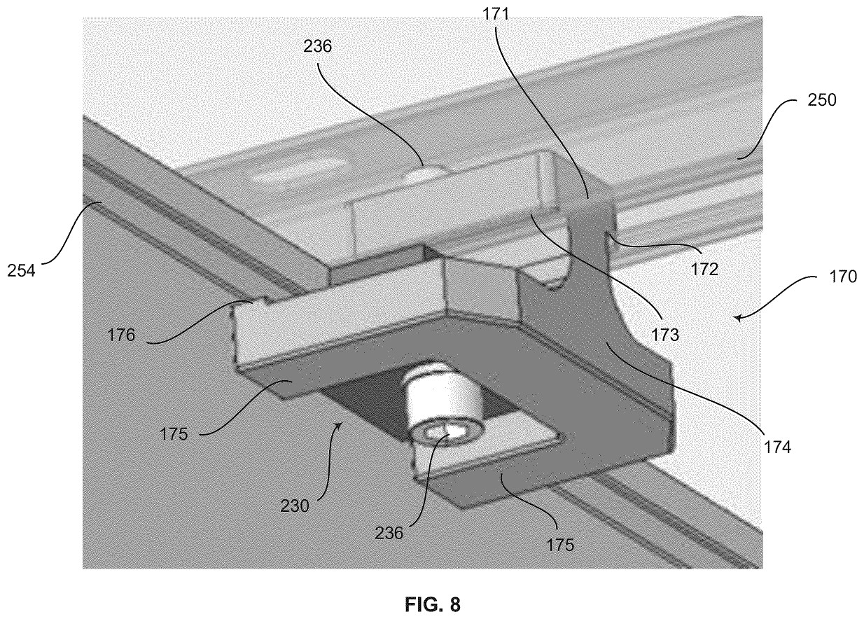

FIG. 6 is a schematic diagram of a lateral view of a ceiling rail 252 mounted to a strut 250 of a ceiling grid by the ceiling rail safety hook 170 and rail fixed nut 230 identified in FIG. 4, in accordance with various embodiments. FIG. 7 is a schematic diagram of a top perspective view of a ceiling rail 252 mounted to a strut 250 of a ceiling grid by a ceiling rail safety hook 170 and rail fixed nut 230, in accordance with various embodiments. FIG. 8 is a schematic diagram of a bottom perspective view of a ceiling rail 252 mounted to a strut 250 of a ceiling grid by a ceiling rail safety hook 170 and rail fixed nut 230, in accordance with various embodiments. Referring to FIGS. 6-8, a ceiling rail 252 is shown mounted to a strut 250 of a ceiling grid. The ceiling rail 252 comprises an upper protrusion 254 extending horizontally outward at the top of the ceiling rail 252. The ceiling rail 252 may be secured substantially flush with a ceiling by rail fixed nuts 230, mounting screws 236, and ceiling rail safety hooks 170. The struts 250 are provided in the ceiling in a grid pattern. The struts 250, such as UNISTRUT or any suitable struts, comprise channels configured to receive fixture mountings, such as medical imaging system fixture mountings, lighting fixture mountings, and/or any suitable fixture mountings. The struts 250 are arranged with the channel facing the floor such that fixture mountings may be inserted into the channel and supported by a lip within the channel of the strut 250. The rail fixed nut 230 comprises a fixed nut body 232 and a fixed nut holder 234. The fixed nut holder 234 extends from the fixed nut body 232 to hold a bottom side of the upper protrusion 254 of the ceiling rail 252.

The ceiling rail safety hook 170 may comprise steel and/or any suitable high strength metal. The ceiling rail safety hook 170 comprises an upper mount 171 and a lower mount 174 extending vertically below the upper mount 171. The upper mount 171 and lower mount 174 generally form an upside down “T” shape when viewed from the front or rear. The upper mount 171 comprises upper mount ledges 172 extending horizontally outward from opposite sides of the upper mount 171. The outer ends of upper mount ledges 172 comprise upper ledge projections 173, which extend downward from outer ends of the upper mount ledges 172. The upper mount ledges 172 and upper ledge projections 173 form hooks that couple with the lips inside the channel of the strut 250. The lower mount 174 extends downward from the upper mount 171 positioned in the channel of the strut 250 and along an outer side of the fixed nut body 232. The lower mount 174 comprises lower mount ledges 175 that extend horizontally outward in a same direction, such that the lower mount 174 and lower mount ledges 175 generally form a “U” shape in a bottom-up view as shown in FIG. 8, for example. The lower mount 174 and lower mount ledges 175 are positioned to generally surround the rail fixed nut 230. The lower mount ledges 175 comprise lower ledge projections 176 extending upward from the ends of the lower mount ledges 175. The lower ledge projections 176 are configured to extend below the upper protrusion 254 of the ceiling rail 252.

A mounting screw 236 is provided and extends through the fixed nut body 232 and through the upper mount 171 of the ceiling safety hook 170. In an exemplary embodiment, the mounting screw 236 may be threaded and secured with a nut and washer. Alternatively, the mounting screw 236 may be secured with a clip ring or any suitable attachment mechanism. In various embodiments,, the lips in the channel of the strut 250 support the upper mount 171 of the ceiling rail safety hook 170. The upper mount 171 of the ceiling rail safety hook 170 supports the mounting screw 236. The mounting screw 236 supports the rail fixed nut 230. The rail fixed nut 230 supports and holds the upper protrusion 254 of the ceiling rail 252 such that the ceiling rail 252 is held against the ceiling. In a normal condition, there is a gap 177 between the lower ledge projections 176 of the lower mount 174 of the ceiling rail safety hook 170 and the upper protrusion 254 of the ceiling rail 252. In a failure condition (i.e., if the mounting screw 236 and/or the rail fixed nut 230 break/fail), the lower ledge projection 176 of the lower mount 174 of the ceiling rail safety hook 170 catches the upper protrusion 254 of the ceiling rail 252 (i.e., no more gap 177) and prevents the ceiling rail 252 from falling from the ceiling. Put another way, in a normal condition, the lower mount 174 of the ceiling rail safety hook 170 does not carry the load of the ceiling rail 252, upper ceiling mount 140, lower ceiling mount 146, and medical imaging system (e.g., X-ray imaging system 100). Instead, the lower mount 174 of the ceiling rail safety hook 170 only carries the load of the ceiling rail 252, upper ceiling mount 140, lower ceiling mount 146, and medical imaging system in a failure condition, such as if the mounting screw 236 and/or rail fixed nut 230 breaks or otherwise fails.

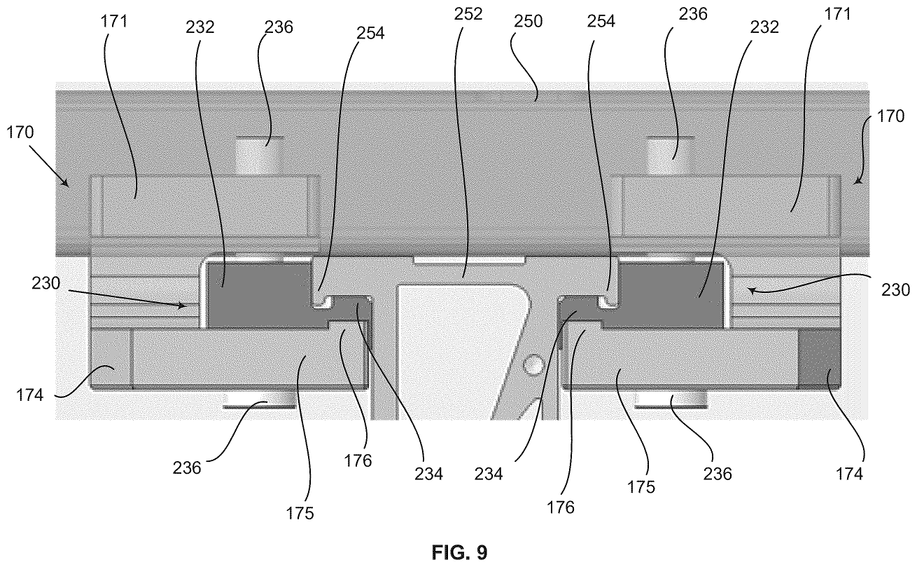

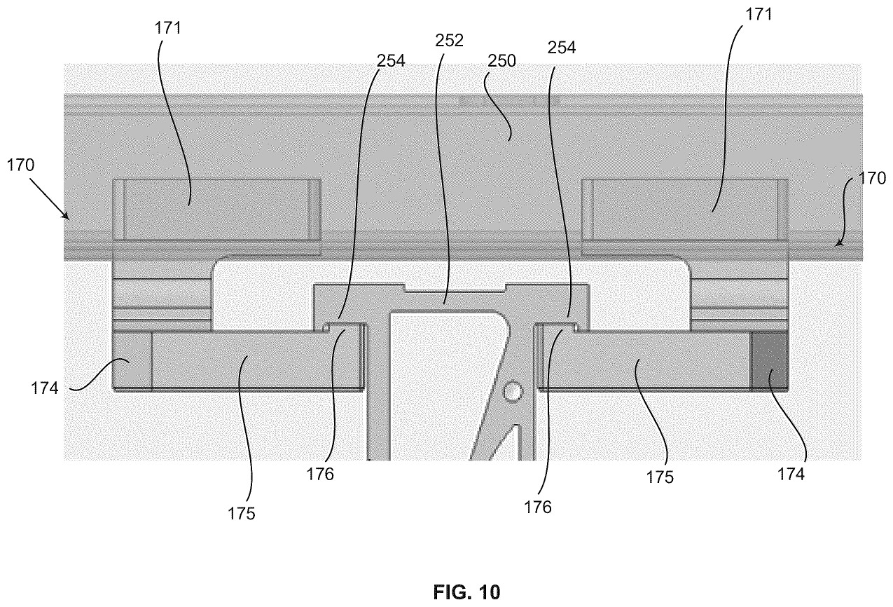

FIG. 9 is a schematic diagram of a lateral view of ceiling rail safety hooks 170 with rail fixed nuts 230 and mounting screws 236 provided on opposite sides of a ceiling rail 252 to mount the ceiling rail 252 to a strut 250 of a ceiling grid in a normal condition, in accordance with various embodiments. FIG. 10 is a schematic diagram of a lateral view of ceiling rail safety hooks 170 provided on opposite sides of a ceiling rail 252 to prevent the ceiling rail 252 from falling from a strut 250 of a ceiling grid after failure of the rail fixed nuts 230 and/or mounting screws 236 shown in FIG. 9, in accordance with various embodiments. Referring to FIGS. 9 and 10, a ceiling rail 252 is shown mounted to a strut 250 of a ceiling grid by rail fixed nuts 230, mounting screws 236, and ceiling safety hooks 170 provided on both sides of the ceiling rail 252. The ceiling rail 252 comprises an upper protrusion 254 extending horizontally outward from both sides of the ceiling rail 252 at the top of the ceiling rail 252. The struts 250 are arranged with the channel open towards the ground such that the ceiling safety hooks 170 and mounting screws 236 may be inserted into the channel and supported by a lip within the channel of the strut 250.

The ceiling rail safety hooks 170 comprise an upper mount 171 and a lower mount 174 extending vertically below the upper mount 171. The upper mount 171 and lower mount 174 generally form a “C” shape when viewed from the side. The upper mount 171 engages lips inside the channel of the strut 250 to secure the ceiling rail safety hooks 170 to the strut 250. The lower mount 174 extends downward from the upper mount 171 positioned in the channel of the strut 250 and along an outer side of the fixed nut body 232. The lower mount 174 comprises lower mount ledges 175 that extend horizontally outward in a same direction (i.e., toward the ceiling rail 252). The lower mount 174 and lower mount ledges 175 are positioned to generally surround the rail fixed nut 230. The lower mount ledges 175 comprise lower ledge projections 176 extending upward from the ends of the lower mount ledges 175. The lower ledge projections 176 are configured to extend below the upper protrusion 254 of the ceiling rail 252.

The rail fixed nut 230 comprises a fixed nut body 232 and a fixed nut holder 234. The fixed nut holder 234 extends from the fixed nut body 232 to hold a bottom side of the upper protrusion 254 of the ceiling rail 252. A mounting screw 236 is provided and extends through the fixed nut body 232 and through the upper mount 171 of the ceiling safety hook 170. In this way, the lips in the channel of the strut 250 support the upper mount 171 of the ceiling rail safety hook 170. The upper mount 171 of the ceiling rail safety hook 170 supports the mounting screw 236. The mounting screw 236 supports the rail fixed nut 230. The rail fixed nut 230 supports and holds the upper protrusion 254 of the ceiling rail 252 such that the ceiling rail 252 is held against the ceiling. In a normal condition as shown in FIG. 9, there is a gap between the lower ledge projections 176 of the lower mount 174 of the ceiling rail safety hook 170 and the upper protrusion 254 of the ceiling rail 252. In a failure condition (i.e., if the mounting screw 236 and/or the rail fixed nut 230 break/fail) as shown in FIG. 10, the lower ledge projection 176 of the lower mount 174 of the ceiling rail safety hook 170 catches the upper protrusion 254 of the ceiling rail 252 and prevents the ceiling rail 252 from falling from the ceiling. In other words, in a normal condition, the lower mount 174 of the ceiling rail safety hook 170 does not carry the load of the ceiling rail 252. Instead, the lower mount 174 of the ceiling rail safety hook 170 only carries the load of the ceiling rail 252 in a failure condition, such as if the mounting screw 236 and/or rail fixed nut 230 breaks or otherwise fails as shown in FIG. 10.

FIG. 11 is a schematic diagram of a perspective view of a portion of an upper ceiling mount having a chassis beams 245 slidably mounted to ceiling rails 252 of a rail system 144 by linear guide assemblies 240 having chassis safety hooks, in accordance with various embodiments. Referring to FIG. 11, two parallel ceiling rails 252 of a rail system 144 of an upper ceiling mount are shown. As discussed above, the ceiling rails 252 may be mounted to struts of a ceiling grid by rail fixed nuts and mounting screws. The ceiling rails 252 comprise upper protrusions 254 and lower protrusions 256. The upper protrusions 252 extend horizontally outward from both sides of the ceiling rails 252 at the top of the ceiling rails 252. The lower protrusions 256 extend from interior sides of the two parallel ceiling rails 252 (i.e., the side of the ceiling rail 252 facing the other ceiling rail 252). Chassis beams 242 of a chassis of an upper ceiling mount are slidably coupled to the lower protrusions 256 of the ceiling rails 252 by linear guide assemblies 240. The linear guide assemblies 240 comprise rollers operable to slidably engage with the lower protrusions 256 of the ceiling rails 252. In a preferred embodiment, the linear guide assemblies 240 comprise chassis safety hooks to prevent the chassis from falling from the ceiling rails 252 of the rail system 144 if the rollers fail (i.e., a failure condition).

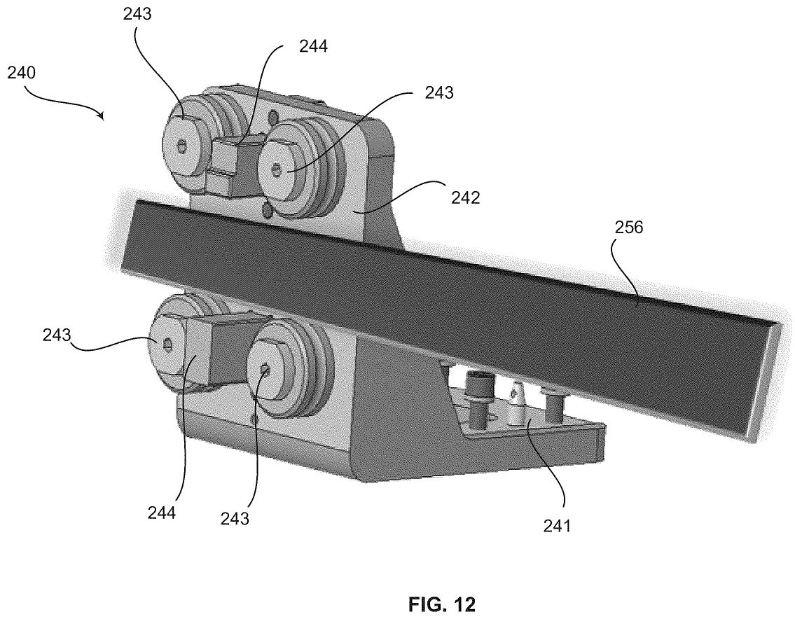

FIG. 12 is a schematic diagram of a perspective view of a linear guide assembly 240 slidably attached to a lower protrusion 256 of a ceiling rail by rollers 243, the linear guide assembly 240 having chassis safety hooks 244, in accordance with various embodiments. The linear guide assembly 240 of FIG. 12 may share various characteristic with the linear guide assemblies 240 discussed above with respect to FIGS. 3, 4, and 11. Referring to FIG. 12, a linear guide assembly 240 is shown. The linear guide assembly 240 may comprise steel and/or any suitable high strength metal. The linear guide assembly 240 comprises a chassis mounting base 241, a rail mounting arm 242, rollers 243, and chassis safety hooks 244, among other things. The chassis mounting base 240 may be configured to be mounted to a chassis, such as the chassis beams 242 of a chassis illustrated in FIG. 11. For example, the chassis mounting base 240 may be mounted to the chassis with mounting screws and/or any suitable attachment mechanism. Referring again to FIG. 12, the rail mounting arm 242 may extend from the chassis mounting base 241 and may be substantially perpendicular to the chassis mounting base 241. In various embodiments, the chassis mounting base 241 and rail mounting arm 242 may be a single integrated piece that is generally “L” shaped when viewed from the side.

The rail mounting arm 242 may comprise a first, interior side facing a chassis and a second, exterior side facing the ceiling rail to which the linear guide assembly 240 is slidably coupled. The second, exterior side of the rail mounting arm 242 comprises at least one pair of rollers 243 configured to slidably receive a lower protrusion 256 of a ceiling rail between each pair of rollers 243. For example, each pair of rollers 243 comprises a top roller 243 and a bottom roller 243 where the top side of the bottom roller 243 slidably engages a bottom side of the lower protrusion 256 and the bottom side of the top roller 243 slidably engages the top side of the lower protrusion 256 such that the linear guide assembly 240 is operable to linearly traverse (i.e., slide across) the lower protrusion 256 in a horizontal direction with respect to a ceiling and floor. Although two pairs of rollers 243 are shown in FIG. 12, more or less pairs of rollers 243 are contemplated (e.g., 1 pair, 3 pairs, 4 pairs, or any suitable number of pairs). Each of the rollers 243 may be formed by a shaft and wheel member that is operable to rotate about the axis of the shaft in either direction.

The second, exterior side of the rail mounting arm 242 further comprises at least one pair of chassis safety hooks 244 extending horizontally outward from the rail mounting arm 242 toward the ceiling rail. The pair of chassis safety hooks 244 comprises a top chassis safety hook 244 configured to be positioned above a top side of the lower protrusion 256 of the ceiling rail and a bottom chassis safety hook 244 configured to be positioned below a bottom side of the lower protrusion 256 of the ceiling rail. The chassis safety hooks 244 are configured to catch the lower protrusion 256 of the ceiling rail in a failure condition (e.g., if one or more of the rollers 243 break and/or otherwise fail). For example, if the rollers 243 fail and the linear guide assembly 240 begins to fall downward, a bottom side of the top chassis safety hook 244 would engage the top side of the lower protrusion 256 to prevent the linear guide assembly 240 from continuing to fall. Moreover, if the linear guide assembly 240 began twisting, a top side of the bottom chassis safety hook 244 would engage the bottom side of the lower protrusion 256 in addition to the bottom side of the top chassis safety hook 244 engaging the top side of the lower protrusion 256. Although one pair of chassis safety hooks 244 is shown in FIG. 12, additional pairs of chassis safety hooks 244 are contemplated (e.g., 2 pairs, 3 pairs, 4 pairs, or any suitable number of pairs). Each of the chassis safety hooks 244 may be a post, shaft, and/or any suitable projection that is operable to catch the lower protrusion 256 of the ceiling rail in the failure condition (e.g., if one or more of the rollers 243 break and/or otherwise fail).

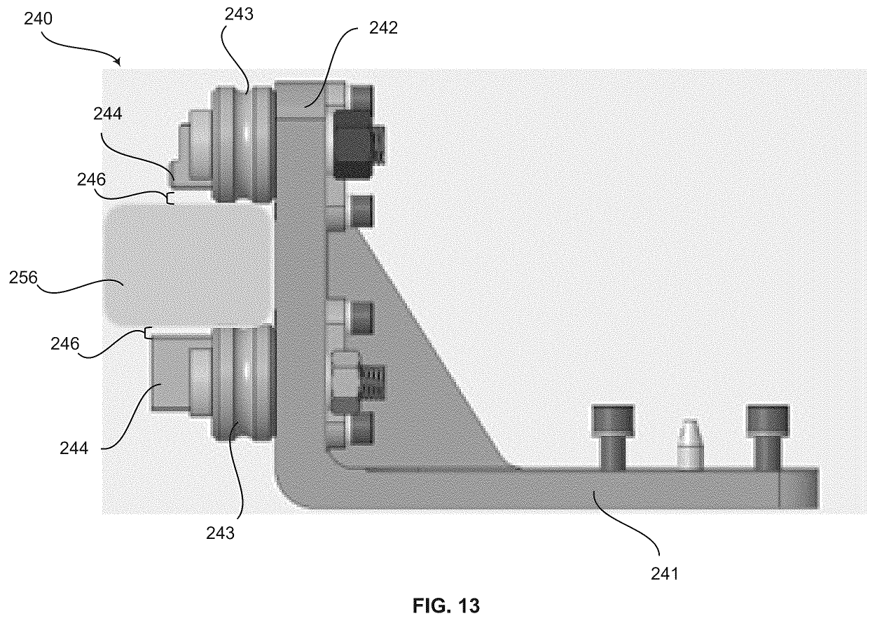

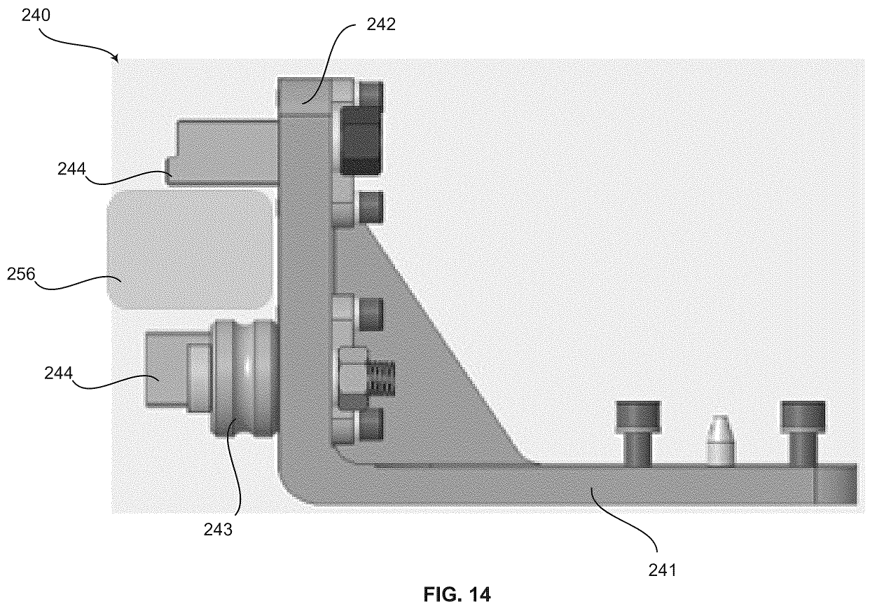

FIG. 13 is a schematic diagram of a lateral view of a linear guide assembly 240 slidably attached to a lower protrusion 256 of a ceiling rail by rollers 243 in a normal condition, the linear guide assembly 240 having chassis safety hooks 244, in accordance with various embodiments. FIG. 14 is a schematic diagram of a lateral view of a linear guide assembly 240 having chassis safety hooks 244 to prevent a chassis attached to the linear guide assembly 240 from falling from the ceiling rail after failure of the upper rollers 243 shown in FIG. 13, in accordance with various embodiments. The linear guide assembly 240 of FIGS. 13 and 14 may share various characteristic with the linear guide assemblies 240 discussed above with respect to FIGS. 3, 4, 11, and 12. Referring to FIGS. 13 and 14, a linear guide assembly 240, which may comprise steel and/or any suitable high strength metal is shown. The linear guide assembly 240 comprises a chassis mounting base 241, a rail mounting arm 242, and chassis safety hooks 244, among other things. The chassis mounting base 240 is operable to be mounted to a chassis with mounting screws and/or any suitable attachment mechanism. The rail mounting arm 242 extends from the chassis mounting base 241 and is substantially perpendicular to the chassis mounting base 241 to form an “L” shape when viewed from the side.

The rail mounting arm 242 comprises a first, interior side facing a chassis and a second, exterior side facing the ceiling rail to which the linear guide assembly 240 is slidably coupled. In a normal condition, the second, exterior side of the rail mounting arm 242 comprises rollers 243 configured to slidably receive a lower protrusion 256 of a ceiling rail. The rollers 243 may be formed by a shaft and wheel member that is operable to rotate about the axis of the shaft in either direction. The second, exterior side of the rail mounting arm 242 further comprises chassis safety hooks 244 extending horizontally outward from the rail mounting arm 242 toward the ceiling rail. The chassis safety hooks 244 comprise a top chassis safety hook 244 configured to be positioned above a top side of the lower protrusion 256 of the ceiling rail and a bottom chassis safety hook 244 configured to be positioned below a bottom side of the lower protrusion 256 of the ceiling rail. The chassis safety hooks 244 are configured to catch the lower protrusion 256 of the ceiling rail in a failure condition (e.g., if one or more of the rollers 243 break and/or otherwise fail) as shown in FIG. 14. For example, if some of the rollers 243 shown in FIG. 13 fail and the linear guide assembly 240 begins to fall downward, a bottom side of the top chassis safety hook 244 engages the top side of the lower protrusion 256 to prevent the linear guide assembly 240 from continuing to fall as shown in FIG. 14. The chassis safety hooks 244 may be posts, shafts, and/or any suitable projection that is operable to catch the lower protrusion 256 of the ceiling rail in the failure condition (e.g., if one or more of the rollers 243 break and/or otherwise fail).

Although various embodiments described above provide an X-ray imaging system 100 mounted to a ceiling 180 by and upper ceiling mount 140 and a lower ceiling mount 146 where the upper ceiling mount comprises a ceiling safety hook and/or a chassis safety hook, unless so claimed the safety hooks are not limited to use in ceiling mounts 140, 146 for X-ray imaging systems 100, and may be provided in ceiling mounts 140, 146 of any suitable medical imaging systems.

Aspects of the present disclosure provide a ceiling mount assembly 140, 146 operable to mount a medical imaging system 100 to a ceiling 180. The ceiling mount assembly 140, 146 may comprise a first ceiling rail 252, a ceiling rail attachment assembly 170, 230, 236, and a chassis 141. The first ceiling rail 252 may comprise a top side configured to face the ceiling 180, a bottom side opposite the top side, an interior side configured to face a second ceiling rail 252 arranged parallel to the first ceiling rail 252, an exterior side opposite the interior side, an upper protrusion 254 projecting horizontally outward from the top side beyond each of the interior side and the exterior side, and a lower protrusion 256 projecting horizontally outward from the interior side and extending along the interior side between opposite ends of each of the first ceiling rail 234. The ceiling rail attachment assembly 170, 230, 236 may comprise a rail fixed nut 230, a ceiling rail safety hook 170, and a mounting screw 236. The rail fixed nut 230 may comprise a fixed nut body 232 and a fixed nut holder 234. The fixed nut holder 234 may extend from the fixed nut body 232 to contact and hold a bottom side of the upper protrusion 254 of the first ceiling rail 252. The ceiling rail safety hook 170 may comprise an upper mount 171 and a lower mount 174. The upper mount may be operable to be fastened to a strut 250 in the ceiling 180. The lower mount 174 may extend downward from the upper mount 171, surround the rail fixed nut 230, and extend below the bottom side of the upper protrusion 254 of the first ceiling rail 252. The mounting screw 236 may extend through the upper mount 171 and the rail fixed nut 230 to secure the first ceiling rail 252 to the strut 250 in the ceiling 180. The lower mount 174 may be configured to receive the bottom side of the upper protrusion 254 in a failure condition of one or both of the rail fixed nut 230 or the mounting screw 236. The chassis 141 may be coupled to the medical imaging system 100. The chassis may comprise a linear guide assembly 240 operable to slidably attach the chassis 141 to the lower protrusion 256 of the first ceiling rail 252. The linear guide assembly 240 may comprise rollers 243 and a first chassis safety hook 244. The rollers 243 may slidably couple to the lower protrusion 256 of the first ceiling rail 252. The first chassis safety hook 244 may extend above a top side of the lower protrusion 256. The first chassis safety hook 244 may be configured to receive the top side of the lower protrusion 256 in a failure condition of the rollers 243.

In an exemplary embodiment, the ceiling rail attachment assembly 170, 230, 236 is one of a plurality of ceiling rail attachment assemblies 170, 230, 236. The ceiling rail attachment assembly 170, 230, 236 may be provided on the interior side of the first ceiling rail 252. An additional ceiling rail attachment assembly 170, 230, 236 of the plurality of ceiling rail attachment assemblies 170, 230, 236 may be provided on the exterior side of the first ceiling rail 252 directly across the first ceiling rail 252 from the ceiling rail attachment assembly 170, 230, 236. In a representative embodiment, one or both of the ceiling rail safety hook 170 and the first chassis safety hook 244 comprises steel. In various embodiments, the ceiling rail safety hook 170 is an upside-down T shape in a front or rear view, and a C shape in a side view. In certain embodiments, a normal condition of the ceiling rail attachment assembly 170, 230, 236 corresponds with normal connections of the mounting screw 236 to the upper mount 171 and the rail fixed nut 230. The lower mount 174 does not carry a load of the first ceiling rail 252 in the normal condition. The lower mount 174 carries the load of the first ceiling rail 252 in the failure condition of one or both of the rail fixed nut 230 or the mounting screw 236. In an exemplary embodiment, a normal condition of the ceiling rail attachment assembly 170, 230, 236 corresponds with normal connections of the mounting screw 236 to the upper mount 171 and the rail fixed nut 230. A gap 177 may be provided between the lower mount 174 and the bottom side of the upper protrusion 254 in the normal condition. The bottom side of the upper protrusion 254 may move to contact the lower mount 174 and remove the gap 177 in the failure condition of one or both of the rail fixed nut 230 or the mounting screw 236. In a representative embodiment, the upper mount 171 comprises upper mount ledges 172 and upper ledge projections 173. The upper mount ledges 172 may extend horizontally outward in opposite directions from opposite sides of an upper mount body 171. The upper ledge projections 173 may extend downward from outer ends of the upper mount ledges 172. The upper mount ledges 172 and upper ledge projections 173 may form hooks operable to couple with lips inside a channel of the strut 250. In various embodiments, the lower mount 174 comprises lower mount ledges 175 and lower ledge projections 176. The lower mount ledges 175 may extend horizontally outward in a same direction from opposite sides of a lower mount body 174 to surround the rail fixed nut 230. The lower mount body 174 and lower mount ledges 175 may form a U-shape in a bottom-up view. The lower ledge projections 176 may extend upward from outer ends of the lower mount ledges 175. The lower ledge projections 176 may be positioned below the bottom side of the upper protrusion 254 of the first ceiling rail 252 and may be configured to receive the bottom side of the upper protrusion 254 in a failure condition of one or both of the rail fixed nut 230 or the mounting screw 236.

In certain embodiments, the linear guide assembly 240 may comprise a chassis mounting base 241 and a rail mounting arm 242. The chassis mounting base 241 may be configured for attachment to the chassis 141. The rail mounting arm 242 may extend vertically from the chassis mounting base 241. The rail mounting arm 242 may comprise an interior side facing the chassis 141 and an exterior side facing the first ceiling rail 252. The rollers 243 and first chassis safety hook 244 may be provided on the exterior side of the rail mounting arm 242. In an exemplary embodiment, the rollers 243 may comprise at least one pair of rollers 243 including a top roller 243 operable to slidably engage the top side of the lower protrusion 256 and a bottom roller 243 operable to slidably engage a bottom side of the lower protrusion 256. The first chassis safety hook 244 may be positioned on the exterior side of the rail mounting arm 242 adjacent the top roller 243. The linear guide assembly 240 may comprise a second chassis safety hook 244 extending below a bottom side of the lower protrusion 256 adjacent the bottom roller 243. The second chassis safety hook 244 may be configured to engage the bottom side of the lower protrusion 256 in a failure condition of the rollers 243. In a representative embodiment, the chassis mounting base 241 and the rail mounting arm 242 form an L-shape.

Various embodiments provide a ceiling mounted medical imaging system 100 comprising a medical imaging system 100 operable to acquire medical image data, and a ceiling mount assembly 140, 146. The ceiling mount assembly 140, 146 may comprise a first ceiling rail 252, a second ceiling rail 252, a ceiling rail attachment assembly 170, 230, 236, and a chassis 141. The second ceiling rail 252 may be arranged parallel to the first ceiling rail 252. Each of the first ceiling rail 252 and the second ceiling rail 252 may comprise a top side facing a ceiling 180, a bottom side opposite the top side, an interior side facing an other of the first ceiling 252 or the second ceiling rail 252, and an exterior side opposite the interior side. The top side may comprise an upper protrusion 254 projecting horizontally outward beyond each of the interior side and the exterior side. The interior side may comprise a lower protrusion 256 extending across the interior side. The ceiling rail attachment assembly 170, 230, 236 may comprise a rail fixed nut 230, a ceiling rail safety hook 170, and a mounting screw 236. The rail fixed nut 230 may be positioned to contact and hold a bottom side of the upper protrusion 254 of the first ceiling rail 252. The ceiling rail safety hook 170 may comprise an upper mount 171 operable to be fastened to a strut 250 in the ceiling 180, and a lower mount 174 extending downward from the upper mount 171. The upper mount 171 may comprise an upper mount body 171, upper mount ledges 172 extending horizontally outward in opposite directions from opposite sides of the upper mount body 171, and upper ledge projections 173 extending downward from outer ends of the upper mount ledges 172. The upper mount ledges 172 and upper ledge projections 173 may form hooks operable to couple with lips inside a channel of the strut 250. The lower mount 174 may comprise a lower mount body 174, lower mount ledges 175, and lower ledge projections 176. The lower mount ledges 175 may extend horizontally outward in a same direction from opposite sides of the lower mount body 174 to surround the rail fixed nut 230. The lower mount body 174 and lower mount ledges 175 may form a U-shape in a bottom-up view. The lower ledge projections 176 may extend upward from outer ends of the lower mount ledges 175. The lower ledge projections 176 may be positioned below the bottom side of the upper protrusion 254 of the first ceiling rail 252. The mounting screw 236 may extend through the upper mount 171 and the rail fixed nut 230 to secure the first ceiling rail 252 to the strut 250 in the ceiling 180. The lower ledge projections 176 may be configured to receive the bottom side of the upper protrusion 254 in a failure condition of one or both of the rail fixed nut 230 or the mounting screw 236. The chassis 141 may be coupled to the medical imaging system 100. The chassis 141 may comprise a linear guide assembly 240 operable to slidably attach the chassis 141 to the lower protrusion 256 of the first ceiling rail 252. The linear guide assembly 240 may comprise rollers 243 slidably coupled to the lower protrusion 256 of the first ceiling rail 252, and a first chassis safety hook 244 extending above a top side of the lower protrusion 256. The first chassis safety hook 244 may be configured to receive the top side of the lower protrusion 256 in a failure condition of the rollers 243.

In a representative embodiment, the linear guide assembly 240 comprises a chassis mounting base 241 configured for attachment to the chassis 141 and a rail mounting arm 242 extending vertically from the chassis mounting base 241. The rail mounting arm 242 may comprise an interior side facing the chassis 141 and an exterior side facing the ceiling rail 252. The rollers 243 and first chassis safety hook 244 may be provided on the exterior side of the rail mounting arm 242. In certain embodiments, the rollers 243 comprise at least one pair of rollers 243 including a top roller 243 operable to slidably engage the top side of the lower protrusion 256 and a bottom roller 243 operable to slidably engage a bottom side of the lower protrusion 256. The first chassis safety hook 244 may be positioned on the exterior side of the rail mounting arm 242 adjacent the top roller 243. The linear guide assembly 240 may comprise a second chassis safety hook 244 extending below a bottom side of the lower protrusion 256 adjacent the bottom roller 243. The second chassis safety hook 244 may be configured to engage the bottom side of the lower protrusion 256 in a failure condition of the rollers 243. In an exemplary embodiment, the chassis mounting base 241 and the rail mounting arm 242 form an L-shape.

Certain embodiments provide a ceiling mounted medical imaging system 100 comprising a medical imaging system 100 operable to acquire medical image data, and a ceiling mount assembly 140, 146. The ceiling mount assembly 140, 146 may comprise a first ceiling rail 252, a second ceiling rail 252, a ceiling rail attachment assembly 170, 230, 236, and a chassis 141. The second ceiling rail 252 may be arranged parallel to the first ceiling rail 252. Each of the first ceiling rail 252 and the second ceiling rail 252 may comprise a top side facing a ceiling 180, a bottom side opposite the top side, an interior side facing an other of the first ceiling rail 252 or the second ceiling rail 252, and an exterior side opposite the interior side. The top side may comprise an upper protrusion 254 projecting horizontally outward beyond each of the interior side and the exterior side. The interior side may comprise a lower protrusion 256 extending across the interior side. The ceiling rail attachment assembly 170, 230, 236 may comprise a rail fixed nut 230, a ceiling rail safety hook 170, and a mounting screw 236. The rail fixed nut 230 may be positioned to contact and hold a bottom side of the upper protrusion 254 of the first ceiling rail 252. The ceiling rail safety hook 170 may comprise an upper mount 171 operable to be fastened to a strut 250 in the ceiling 180 and a lower mount 174 extending downward from the upper mount 171, surrounding the rail fixed nut 230, and extending below the bottom side of the upper protrusion 254 of the first ceiling rail 252. The mounting screw 236 may extend through the upper mount 171 and the rail fixed nut 230 to secure the first ceiling rail 252 to the strut 250 in the ceiling 180. The lower mount 174 may be configured to receive the bottom side of the upper protrusion 254 in a failure condition of one or both of the rail fixed nut 230 or the mounting screw 236. The chassis 141 may be coupled to the medical imaging system 100. The chassis 141 may comprise a linear guide assembly 240 operable to slidably attach the chassis 141 to the lower protrusion 256 of the first ceiling rail 252. The linear guide assembly 240 may comprises a chassis mounting base 241, a rail mounting arm 242, rollers 243, a first chassis safety hook 244, and a second chassis safety hook 244. The chassis mounting base 241 may be configured for attachment to the chassis 141. The rail mounting arm 242 may extend vertically from the chassis mounting base 241. The rail mounting arm 242 may comprise an interior side facing the chassis 141 and an exterior side facing the first ceiling rail 252. The rollers 243 may slidably couple to the lower protrusion 256 of the first ceiling rail 252. The rollers 243 may comprise at least one pair of rollers 243 including a top roller 243 operable to slidably engage the top side of the lower protrusion 256, and a bottom roller operable to slidably engage a bottom side of the lower protrusion 256. The first chassis safety hook 244 may be positioned on the exterior side of the rail mounting arm 242 adjacent the top roller 243 and extending above a top side of the lower protrusion 256. The first chassis safety hook 244 may be configured to receive the top side of the lower protrusion 256 in a failure condition of the rollers 243. The second chassis safety hook 244 may be positioned on the exterior side of the rail mounting arm 242 adjacent the bottom roller 243 and extending below a bottom side of the lower protrusion 256. The second chassis safety hook 244 may be configured to engage the bottom side of the lower protrusion 256 in a failure condition of the rollers 243.

In various embodiments, the ceiling rail safety hook 170 may be an upside-down T shape in a front or rear view and a C shape in a side view. In an exemplary embodiment, a normal condition of the ceiling rail attachment assembly 170, 230, 236 corresponds with normal connections of the mounting screw 236 to the upper mount 171 and the rail fixed nut 230. A gap 177 may be provided between the lower mount 174 and the bottom side of the upper protrusion 254 in the normal condition. The bottom side of the upper protrusion 254 may move to contact the lower mount 174 and remove the gap 177 in the failure condition of one or both of the rail fixed nut 230 or the mounting screw 236. In a representative embodiment, the upper mount comprises upper mount ledges 172 extending horizontally outward in opposite directions from opposite sides of an upper mount body 171, and upper ledge projections 173 extending downward from outer ends of the upper mount ledges 172. The upper mount ledges 172 and upper ledge projections 173 may form hooks operable to couple with lips inside a channel of the strut 250.

In certain embodiments, the lower mount 174 comprises lower mount ledges 175 and lower ledge projections 176. The lower mount ledges 175 may extend horizontally outward in a same direction from opposite sides of a lower mount body 174 to surround the rail fixed nut 230. The lower mount body 174 and lower mount ledges 175 may form a U-shape in a bottom-up view. The lower ledge projections 176 may extend upward from outer ends of the lower mount ledges 175. The lower ledge projections 176 may be positioned below the bottom side of the upper protrusion 254 of the first ceiling rail 252 and configured to receive the bottom side of the upper protrusion 254 in a failure condition of one or both of the rail fixed nut 230 or the mounting screw 236.

As utilized herein the term “circuitry” refers to physical electronic components (i.e., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a component is “operable” or “configured” to perform a function whenever the component comprises the necessary structure to perform the function, regardless of whether the function is performed.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A ceiling mount assembly operable to mount a medical imaging system to a ceiling, the ceiling mount assembly comprising:
a first ceiling rail comprising:
a top side configured to face the ceiling;
a bottom side opposite the top side;
an interior side configured to face a second ceiling rail arranged parallel to the first ceiling rail;
an exterior side opposite the interior side;
an upper protrusion projecting horizontally outward from the top side beyond each of the interior side and the exterior side; and
a lower protrusion projecting horizontally outward from the interior side and extending along the interior side between opposite ends of each of the first ceiling rail;
a ceiling rail attachment assembly comprising:
a rail fixed nut comprising a fixed nut body and a fixed nut holder, wherein the fixed nut holder extends from the fixed nut body to contact and hold a bottom side of the upper protrusion of the first ceiling rail;
a ceiling rail safety hook comprising:
an upper mount operable to be fastened to a strut in the ceiling; and
a lower mount extending downward from the upper mount, surrounding the rail fixed nut, and extending below the bottom side of the upper protrusion of the first ceiling rail; and
a mounting screw extending through the upper mount and the rail fixed nut to secure the first ceiling rail to the strut in the ceiling, wherein the lower mount is configured to receive the bottom side of the upper protrusion in a failure condition of one or both of the rail fixed nut or the mounting screw;
a chassis coupled to the medical imaging system, the chassis comprising a linear guide assembly operable to slidably attach the chassis to the lower protrusion of the first ceiling rail, wherein the linear guide assembly comprises:
rollers slidably coupled to the lower protrusion of the first ceiling rail; and
a first chassis safety hook extending above a top side of the lower protrusion, wherein the first chassis safety hook is configured to receive the top side of the lower protrusion in a failure condition of the rollers.

2. The ceiling mount assembly of claim 1, wherein:
the ceiling rail attachment assembly is one of a plurality of ceiling rail attachment assemblies;
the ceiling rail attachment assembly is provided on the interior side of the first ceiling rail; and
an additional ceiling rail attachment assembly of the plurality of ceiling rail attachment assemblies is provided on the exterior side of the first ceiling rail directly across the first ceiling rail from the ceiling rail attachment assembly.

3. The ceiling mount assembly of claim 1, wherein one or both of the ceiling rail safety hook and the first chassis safety hook comprises steel.

4. The ceiling mount assembly of claim 1, wherein the ceiling rail safety hook is:
an upside-down T shape in a front or rear view; and
a C shape in a side view.

5. The ceiling mount assembly of claim 1, wherein:
a normal condition of the ceiling rail attachment assembly corresponds with normal connections of the mounting screw to the upper mount and the rail fixed nut;
the lower mount does not carry a load of the first ceiling rail in the normal condition; and
the lower mount carries the load of the first ceiling rail in the failure condition of one or both of the rail fixed nut or the mounting screw.

6. The ceiling mount assembly of claim 1, wherein:
a normal condition of the ceiling rail attachment assembly corresponds with normal connections of the mounting screw to the upper mount and the rail fixed nut;
a gap is provided between the lower mount and the bottom side of the upper protrusion in the normal condition; and
the bottom side of the upper protrusion moves to contact the lower mount and removes the gap in the failure condition of one or both of the rail fixed nut or the mounting screw.

7. The ceiling mount assembly of claim 1, wherein the upper mount comprises:
upper mount ledges extending horizontally outward in opposite directions from opposite sides of an upper mount body; and
upper ledge projections extending downward from outer ends of the upper mount ledges, the upper mount ledges and upper ledge projections forming hooks operable to couple with lips inside a channel of the strut.

8. The ceiling mount assembly of claim 1, wherein the lower mount comprises:
lower mount ledges extending horizontally outward in a same direction from opposite sides of a lower mount body to surround the rail fixed nut, wherein the lower mount body and lower mount ledges form a U-shape in a bottom-up view; and
lower ledge projections extending upward from outer ends of the lower mount ledges, the lower ledge projections positioned below the bottom side of the upper protrusion of the first ceiling rail and configured to receive the bottom side of the upper protrusion in a failure condition of one or both of the rail fixed nut or the mounting screw.

9. The ceiling mount assembly of claim 1, wherein the linear guide assembly comprises:
a chassis mounting base configured for attachment to the chassis; and
a rail mounting arm extending vertically from the chassis mounting base, the rail mounting arm comprising an interior side facing the chassis and an exterior side facing the first ceiling rail, wherein the rollers and first chassis safety hook are provided on the exterior side of the rail mounting arm.

10. The ceiling mount assembly of claim 9, wherein:
the rollers comprise at least one pair of rollers including:
a top roller operable to slidably engage the top side of the lower protrusion; and
a bottom roller operable to slidably engage a bottom side of the lower protrusion;
the first chassis safety hook is positioned on the exterior side of the rail mounting arm adjacent the top roller; and
the linear guide assembly comprises a second chassis safety hook extending below a bottom side of the lower protrusion adjacent the bottom roller, wherein the second chassis safety hook is configured to engage the bottom side of the lower protrusion in a failure condition of the rollers.

11. The ceiling mount assembly of claim 9, wherein the chassis mounting base and the rail mounting arm form an L-shape.

12. A ceiling mounted medical imaging system, comprising:
a medical imaging system operable to acquire medical image data; and
a ceiling mount assembly comprising:
a first ceiling rail and a second ceiling rail arranged parallel to the first ceiling rail, each of the first ceiling rail and the second ceiling rail comprising a top side facing a ceiling, a bottom side opposite the top side, an interior side facing an other of the first ceiling rail or the second ceiling rail, and an exterior side opposite the interior side, wherein the top side comprises an upper protrusion projecting horizontally outward beyond each of the interior side and the exterior side, and wherein the interior side comprises a lower protrusion extending across the interior side;
a ceiling rail attachment assembly comprising:
a rail fixed nut positioned to contact and hold a bottom side of the upper protrusion of the first ceiling rail;
a ceiling rail safety hook comprising:
an upper mount operable to be fastened to a strut in the ceiling, the upper mount comprising:
an upper mount body;
upper mount ledges extending horizontally outward in opposite directions from opposite sides of the upper mount body; and
upper ledge projections extending downward from outer ends of the upper mount ledges, the upper mount ledges and upper ledge projections forming hooks operable to couple with lips inside a channel of the strut; and
a lower mount extending downward from the upper mount, the lower mount comprising:
a lower mount body;
lower mount ledges extending horizontally outward in a same direction from opposite sides of the lower mount body to surround the rail fixed nut, wherein the lower mount body and lower mount ledges form a U-shape in a bottom-up view; and
lower ledge projections extending upward from outer ends of the lower mount ledges, the lower ledge projections positioned below the bottom side of the upper protrusion of the first ceiling rail; and
a mounting screw extending through the upper mount and the rail fixed nut to secure the first ceiling rail to the strut in the ceiling, wherein the lower ledge projections are configured to receive the bottom side of the upper protrusion in a failure condition of one or both of the rail fixed nut or the mounting screw; and
a chassis coupled to the medical imaging system, the chassis comprising a linear guide assembly operable to slidably attach the chassis to the lower protrusion of the first ceiling rail, wherein the linear guide assembly comprises:
rollers slidably coupled to the lower protrusion of the first ceiling rail; and
a first chassis safety hook extending above a top side of the lower protrusion, wherein the first chassis safety hook is configured to receive the top side of the lower protrusion in a failure condition of the rollers.

13. The ceiling mounted medical imaging system of claim 12, wherein the linear guide assembly comprises:
a chassis mounting base configured for attachment to the chassis; and
a rail mounting arm extending vertically from the chassis mounting base, the rail mounting arm comprising an interior side facing the chassis and an exterior side facing the ceiling rail, wherein the rollers and first chassis safety hook are provided on the exterior side of the rail mounting arm.

14. The ceiling mounted medical imaging system of claim 13, wherein:
the rollers comprise at least one pair of rollers including:
a top roller operable to slidably engage the top side of the lower protrusion; and
a bottom roller operable to slidably engage a bottom side of the lower protrusion;
the first chassis safety hook is positioned on the exterior side of the rail mounting arm adjacent the top roller; and
the linear guide assembly comprises a second chassis safety hook extending below a bottom side of the lower protrusion adjacent the bottom roller, wherein the second chassis safety hook is configured to engage the bottom side of the lower protrusion in a failure condition of the rollers.

15. The ceiling mounted medical imaging system of claim 13, wherein the chassis mounting base and the rail mounting arm form an L-shape.

16. A ceiling mounted medical imaging system, comprising:
a medical imaging system operable to acquire medical image data; and
a ceiling mount assembly comprising:
a first ceiling rail and a second ceiling rail arranged parallel to the first ceiling rail, each of the first ceiling rail and the second ceiling rail comprising a top side facing a ceiling, a bottom side opposite the top side, an interior side facing an other of the first ceiling rail or the second ceiling rail, and an exterior side opposite the interior side, wherein the top side comprises an upper protrusion projecting horizontally outward beyond each of the interior side and the exterior side, and wherein the interior side comprises a lower protrusion extending across the interior side;
a ceiling rail attachment assembly comprising:
a rail fixed nut positioned to contact and hold a bottom side of the upper protrusion of the first ceiling rail;
a ceiling rail safety hook comprising:
an upper mount operable to be fastened to a strut in the ceiling; and
a lower mount extending downward from the upper mount, surrounding the rail fixed nut, and extending below the bottom side of the upper protrusion of the first ceiling rail; and
a mounting screw extending through the upper mount and the rail fixed nut to secure the first ceiling rail to the strut in the ceiling, wherein the lower mount is configured to receive the bottom side of the upper protrusion in a failure condition of one or both of the rail fixed nut or the mounting screw;
a chassis coupled to the medical imaging system, the chassis comprising a linear guide assembly operable to slidably attach the chassis to the lower protrusion of the first ceiling rail, wherein the linear guide assembly comprises:
a chassis mounting base configured for attachment to the chassis;
a rail mounting arm extending vertically from the chassis mounting base, the rail mounting arm comprising an interior side facing the chassis and an exterior side facing the first ceiling rail;
rollers slidably coupled to the lower protrusion of the first ceiling rail, the rollers comprising at least one pair of rollers including:
a top roller operable to slidably engage the top side of the lower protrusion; and
a bottom roller operable to slidably engage a bottom side of the lower protrusion;
a first chassis safety hook positioned on the exterior side of the rail mounting arm adjacent the top roller and extending above a top side of the lower protrusion, wherein the first chassis safety hook is configured to receive the top side of the lower protrusion in a failure condition of the rollers; and
a second chassis safety hook positioned on the exterior side of the rail mounting arm adjacent the bottom roller and extending below a bottom side of the lower protrusion, wherein the second chassis safety hook is configured to engage the bottom side of the lower protrusion in a failure condition of the rollers.

17. The ceiling mounted medical imaging system of claim 16, wherein the ceiling rail safety hook is:
an upside-down T shape in a front or rear view; and
a C shape in a side view.

18. The ceiling mounted medical imaging system of claim 16, wherein:
a normal condition of the ceiling rail attachment assembly corresponds with normal connections of the mounting screw to the upper mount and the rail fixed nut;
a gap is provided between the lower mount and the bottom side of the upper protrusion in the normal condition; and
the bottom side of the upper protrusion moves to contact the lower mount and removes the gap in the failure condition of one or both of the rail fixed nut or the mounting screw.

19. The ceiling mounted medical imaging system of claim 16, wherein the upper mount comprises:
upper mount ledges extending horizontally outward in opposite directions from opposite sides of an upper mount body; and
upper ledge projections extending downward from outer ends of the upper mount ledges, the upper mount ledges and upper ledge projections forming hooks operable to couple with lips inside a channel of the strut.

20. The ceiling mounted medical imaging system of claim 16, wherein the lower mount comprises:
lower mount ledges extending horizontally outward in a same direction from opposite sides of a lower mount body to surround the rail fixed nut, wherein the lower mount body and lower mount ledges form a U-shape in a bottom-up view; and
lower ledge projections extending upward from outer ends of the lower mount ledges, the lower ledge projections positioned below the bottom side of the upper protrusion of the first ceiling rail and configured to receive the bottom side of the upper protrusion in a failure condition of one or both of the rail fixed nut or the mounting screw.
